(19) United States
(12) Patent Application Publication
(10) Pub. No.: US 2023/0329539 A1
(43) Pub. Date: Oct. 19, 2023

(54) THROAT EXAMINATION APPARATUS

(71) Applicant: Nicholas James Ian HAMILTON, London, Greater London (GB)

(72) Inventor: Nicholas James Ian Hamilton, London, Greater London (GB)

(21) Appl. No.: 18/028,805

(22) PCT Filed: Oct. 8, 2021
(86) PCT No.: PCT/EP2021/077963

§ 371 Date: Mar. 28, 2023

(30) Foreign Application Priority Data
Oct. 15, 2020 (GB) 2016391.1
Jul. 23, 2021 (GB) 2110647.1

(51) Int. Cl. A61B 1/267; A61B 1/05; A61B 1/00

(52) U.S. Cl. CPC A61B 1/267; A61B 1/00018; A61B 1/00045; A61B 1/00121; A61B 1/05

(57) ABSTRACT

The present disclosure relates to a throat examination device (1) for examining a throat of a patient. The throat examination device (1) has a support arm (7) having a longitudinal axis (X); and an imaging unit (10). The imaging unit (10) is disposed on the support arm (7) and includes at least one imaging sensor (11-n) for capturing an image of at least a portion of one or more of the oral cavity, the oropharynx and the laryngopharynx. The imaging unit (10) has a transverse angle of view (TAOV) in a transverse plane extending perpendicular to the longitudinal axis (X) of the support arm (7). The imaging unit (10) may be provided in an imaging module (3) which is removably mounted to the support arm (7). A disposable cover (45) may be provided over the imaging unit (10).

### TECHNICAL FIELD

The present disclosure relates to a throat examination apparatus. In particular, but not exclusively, the throat examination apparatus is suitable for capturing an image of an oral cavity and/or throat of a patient.

### BACKGROUND

The examination of the deep part of a patient’s throat, containing a base of the tongue and voice box, is part of a routine examination for patients attending Ear, Nose and Throat (ENT) clinic with throat symptoms. In the majority of cases, this examination is carried out using a flexible fibreoptic camera (flexible endoscopy) that is inserted through the nose and down the back of the throat to provide a live image. In a minority of cases, examination of the vocal cords is carried out by a rigid straight fibreoptic camera inserted through the mouth (rigid laryngoscopy). Whilst fibreoptic endoscopy has revolutionised the way clinicians are able to diagnose and assess diseases in the throat, it has a number of limitations. The first limitation is the dependence on a trained user. The procedure requires training on how to use the endoscope safely and necessitates an understanding of anatomy to guide the endoscope into the correct position. Manual dexterity is also needed to navigate the twists and turns of the upper airway to reach the deep part of the throat. Consequently, only trained specialists in ENT and a small number of allied health professionals are able to perform the investigation. An example of why this is a problem can be seen if you consider a patient who presents to the emergency department with a possible fishbone stuck in their throat. Unless there is an ENT specialist on site, the patient requires transfer to an ENT centre in order for the specialist to perform the examination of the deep part of the throat and confirm or exclude the presence of the fishbone.

The second limitation is the restricted field of view with a fibreoptic device. As fibreoptic cameras work by transmitting light through the fibreoptic channel, the image is unidirectional i.e. you can only visualise what the camera is pointing at. This requires the operator to build up a mental picture of the entire throat by systematically moving from one area to another, a process aided by taking multiple pictures. The operator also needs experience in performing the procedure to understand what represents a true abnormality and what represents a normal variant. To become skilled in this takes many years and until mastered, can lead to missed diagnosis if a diseased area is not noticed or outside the field of view.

Some patients may also experience discomfort on passing a flexible camera through the nose and into the throat. Whilst the use of local anaesthetic spray can limit this, certain patients find the investigation difficult to tolerate regardless of anaesthesia. This is especially true amongst children who generally are not tolerant of the examination and require general anaesthesia if visualising the deep part of the throat is felt to be essential. Some patients also have structural abnormalities in their nose that makes the investigation challenging and often painful.

A further consideration is cost and sterility. Whilst, disposable flexible endoscopes have recently been marketed, the high costs associated with each device prohibits routine use in clinic patients in most modern healthcare systems. Instead, the majority of flexible endoscopes are reusable and require sterilisation between use. This process is in itself costly, time consuming and can be ineffective if performed incorrectly which can place patients at risk.

In general, modern imaging techniques are limited in imaging the upper part of the throat in the home setting. The unidirectional nature of conventional imaging coupled with the difficulties in individuals being able to see inside the throat to know where to point a camera result in individuals being unable to send useful pictures of their throat to doctors as part of a telemedicine consultation for throat disorders such as tonsillitis.

At least in certain embodiments, the present invention seeks to overcome or ameliorate at least some of the limitations associated with prior art systems.

### SUMMARY OF THE INVENTION

Aspects of the present invention relate to a throat examination device as claimed in the appended claims.

According to an aspect of the present invention there is provided a throat examination device for examining a throat of a patient, the throat examination device comprising:
a support arm having a longitudinal axis; and
an imaging unit disposed on the support arm and comprising at least one imaging sensor for capturing an image of at least a portion of one or more of: the oral cavity, the oropharynx and the laryngopharynx;
wherein the imaging unit has a transverse angle of view in a transverse plane extending perpendicular to the longitudinal axis of the support arm.

At least in certain embodiments, the imaging unit may capture an image having a wide transverse angle of view. The imaging unit may capture a panoramic image. The throat examination device is suitable for examining the throat of a patient. At least in certain embodiments, the throat examination device may be used to examine the patient’s oral cavity. The image captured by the throat examination device may provide a comprehensive view of the area(s) of interest in a patient’s oral cavity and/or throat. The throat examination device may be suitable for capturing an image of at least a portion of the hypopharynx. The at least one imaging sensor may be suitable for capturing an image of at least a portion of the laryngopharynx. Alternatively, or in addition, the throat examination device may be suitable for capturing an image of at least a portion of the pharynx and/or at least a portion of the oral cavity. The image may comprise a photographic still image or a video image. By capturing an image having an angle of view which is wider than prior art systems, the chance of a relevant area being missed may be reduced. This may also allow a relatively unskilled health professional to take the image. At least in certain embodiments, a patient may be able to take images of their own throat, for example in their own home, as part of a telemedicine consultation. The throat examination device may be combined with an artificial intelligence technology and used as an automated screening test, for example as part of a cancer surveillance program. A useful analogy to describe the benefits of the extended field of view afforded by the throat examination device described herein can be seen when considering a jigsaw puzzle. The imaging unit provides an image which covers the relevant region within the oral cavity and throat. In contrast, a flexible endoscopy provides a number of jigsaw pieces that must then be assembled to provide a complete picture. However, this cognitive process of combining a plurality of images requires training and experience and may be prone to missing certain pieces that make the picture incomplete.

The imaging unit has a second angle of view in a longitudinal plane extending along the longitudinal axis. At least in certain embodiments, the imaging unit may capture an image having a wide longitudinal angle of view.

The imaging unit may comprise a first field of view. The first and second angles of view define the field of view of the imaging unit. The first field of view may extend around at least a portion of the longitudinal axis.

The imaging unit may comprise or consist of a first lens having a first optical axis. In arrangements in which the first lens is the only lens provided in the imaging unit, the angle of view of the first lens corresponds to the transverse angle of view in the transverse plane.

The imaging unit is suitable for insertion (at least partially) into an oral cavity of the patient. A portion of the support arm, for example a distal end thereof, may be inserted into the patient’s oral cavity.

The transverse angle of view of the imaging unit may be greater than or equal to 180°, or greater than or equal to 235°; or the transverse angle of view may be greater than or equal to 270°. In certain embodiments, the transverse angle of view may be approximately 360°. In certain embodiments, the imaging unit may have a field of view extending through approximately 360°.

The at least one imaging sensor may capture an image comprising a region at least substantially surrounding the imaging unit. The ability to capture an image having a wide angle of view allows an image of a specific area of interest to be captured without requiring the imaging unit is pointed directly at the area of interest. At least in certain embodiments, this may reduce the need for training as the image or video can be taken by simply placing the imaging unit in the oral cavity of the patient, thereby reducing or avoiding the need to navigate the complex anatomy of the nose. Furthermore, at least in certain embodiments, the throat examination device may be used without the operator having to know what they are looking at or what part of the throat is relevant. The imaging unit may capture an image over a sufficient area to enable the relevance of particular areas to be assessed subsequently, for example by a specialist interpreting the image. This enables an image or video to be taken using a single “point and shoot” method rather than the operator needing to focus the camera on multiple areas and taking multiple images.

A typical use case may involve a patient having a suspected fish bone lodged within the throat presenting to an emergency department. The emergency doctor may not have the expertise to examine the deeper part of the throat with a flexible endoscope. Under normal circumstances, the patient would be transferred to a hospital where the relevant expertise was available. The throat examination device described herein may avoid the need to the patient to be transferred. The emergency doctor could acquire an image or video of the throat by placing the throat examination device in the back of the throat via the mouth. The resulting images can then be transferred electronically to an ENT specialist at a different hospital who can review the images on a computer monitor, to exclude or confirm the presence of the fish bone. The procedure may be performed in less than five (5) seconds, for example.

At least a portion of the imaging unit may be located in the oral cavity of the patient. The at least one imaging sensor may be operable to capture an image, which may be a static image or a video image, of an interior of the oral cavity and/or throat. The image may be captured in a digital format and may be stored on a memory device and/or output to a display device.

In use, the throat examination device is positioned in the mouth and activated to acquire the image or video. This is likely to be much better tolerated by a patient than a flexible endoscope which is introduced through the nose. The corresponding procedure for a flexible endoscope may take several minutes to complete, whereas it is envisaged that the required images may be acquired using the throat examination device in less than 5 or 10 seconds. The tolerance of the procedure may be especially important when treating children where the process would be similar to examining the back of the throat with a wooden spatula.

The at least one image sensor may comprise a CCD sensor, for example. The imaging unit may comprise a plurality of the imaging sensors. The at least one imaging unit may combine the images from two or more imaging sensors to generate a composite image. The throat examination device may comprise an electronic processor configured to generate the composite image; or the composite image may be generated by an external processing unit.

The composite image may be formed by stitching together two or more images captured by respective imaging sensors. The images may be joined along common edges to form the composite image. The two or more images may comprise an overlapping image region. The processing of the images may comprise identifying the overlapping image region in two or more images. The overlapping image region may be identified by processing the images to identify common image features or image elements present in each of the images. The images may be aligned with each other in dependence on the identified overlapping image region, for example to align the overlapping image region identified in the two or more images. Alternatively, or in addition, at least one of the two or more images may be cropped or otherwise transformed at least partially to remove the overlapping image region. The image processing may be performed by the electronic processor provided in the throat examination device or an external processing unit.

The imaging unit may comprise a first lens having a first subsidiary transverse angle of view in the transverse plane. The imaging unit may comprise a second lens having a second subsidiary transverse angle of view in the transverse plane. The first lens and the second lens may be associated with a single imaging sensor. For example, the first and second lens may be configured to project light onto discrete regions of the imaging sensor. One or more mirror may be incorporated into the imaging unit to direct light onto the imaging sensor from the first lens and/or the second lens. One or more prism may be incorporated into the imaging unit to direct light onto the imaging sensor from the first lens and/or the second lens Alternatively, first and second lenses may be associated with first and second imaging sensors respectively.

At least one of the first and second lenses may comprise a wide-angle lens or an ultra-wide-angle lens. At least one of the first and second lenses may comprise a fisheye lens. The first and second lenses may each comprise a wide-angle lens or an ultra-wide-angle lens. The first and second lenses may each comprise a so-called fisheye lens.

The first lens may have a first subsidiary transverse angle of view in the range 100° to 180°, inclusive. The first subsidiary transverse angle of view may be approximately 180°. The second lens may have a second subsidiary transverse angle of view in the range 100° to 180°, inclusive. The second subsidiary transverse angle of view may be approximately 180°.

In a variant, the first lens may have a first subsidiary transverse angle of view greater than 180°; and/or the second may have a second subsidiary transverse angle of view greater than 180°. The images captured by the first and second lenses may comprise an overlapping image region. The processing of the images may comprise reducing or removing the overlapping image region. A composite image may be formed which at least partially removes the overlapping image region.

The first and second lenses may be aligned with each other along the longitudinal axis. The first and second lenses may have first and second optical axes which are at least substantially aligned with each other.

Alternatively, the first and second lenses may be offset from each other along the longitudinal axis. An offset may facilitate packaging the first and second lenses in the support arm. The image captured by the imaging unit may be processed to accommodate any such offset, for example by applying a transform to one or both sets of image data.

The imaging unit may comprise a third lens. The third lens may have a third subsidiary transverse angle of view in the transverse plane.

The image generated by the imaging unit may be a composite image formed by combining subsidiary images captured via two or more lenses. The transverse angle of view of the imaging unit may be composed of the first and second subsidiary transverse angles of view. Thus, the combined angles of view of the lenses determines a (cumulative) transverse angle of view of the imaging unit. It will be understood that this is also applicable if there are more than two lenses. The (cumulative) transverse angle of view of the imaging unit may be defined taking account of any overlap between the fields of view of the lenses. Preferably, the lenses are configured to reduce or avoid any overlap.

The first and second lenses may be disposed on a superior (upper) surface and an inferior (lower) surface of the imaging unit.

The first lens may have a first optical axis; and the second lens may have a second optical axis. The first and second optical axes may extend substantially perpendicular to the longitudinal axis. The first and second optical axes may be oriented in opposing directions.

The first and second optical axes may be co-axial, i.e., disposed on a common axis. Alternatively, the first and second optical axes may be angularly offset and/or spatially offset. The spatial offset may be along the longitudinal axis and/or perpendicular to the longitudinal axis. The first and second optical axes may extend parallel to each other. The first and second lenses may face in opposite directions, for example in tangential directions from diametrically opposed positions on the imaging unit. Alternatively, the first and second lenses may face in the same direction, for example in the form of a stereo camera. This arrangement may enable determination of distancing (range) information, for example to determine contours of the oral cavity.

The image captured by the imaging unit may be processed to accommodate any such offset, for example by applying a transform to one or both sets of image data.

The imaging unit may comprise first and second imaging sensors for capturing images. The first imaging sensor being associated with the first lens and the second imaging sensor being associated with the second lens.

The throat examination device may comprise at least one control unit. The at least one control unit may comprise:
an electronic processor having:
   one or more electrical input for receiving image data generated by the at least one imaging sensor; and/or
   one or more electrical output for outputting image data to an external device. one or more electrical output for outputting image data to an external device.

The throat examination device may comprise a user interface for generating an image capture request in dependence on a user input. The user interface may comprise one or more buttons or switches, for example. The at least one control unit may be configured to receive the image capture request. The at least one control unit may be configured to capture image data generated by the at least one imaging sensor in dependence on the image capture request.

The captured image data may comprise a photographic (still) image or a video image. The photographic image may be captured in dependence on a momentary activation of the image capture button, for example comprising depressing and releasing the image capture button in a predetermined time period. The video image may be captured for a period of time while an image capture button is depressed.

The imaging unit may be disposed in an imaging module. The imaging module may be removably mounted on the support arm. The throat examination device may comprise one or more mechanical fastener for releasably fastening the imaging module to the support arm. The or each mechanical fastener may, for example, comprise cooperating male and female fasteners. The male and female fasteners may comprise cooperating threaded fasteners.

The support arm may comprise one or locking features to lock the imaging module in position. Alternatively, or in addition, the imaging module and the support arm may comprise one or more alignment features for aligning the imaging unit in a predetermined orientation. The one or more alignment features may ensure that the imaging module has a predetermined angular orientation when mounted to the support arm. This may facilitate comparison of images captured by the at least one imaging sensor.

The throat examination device may comprise a disposable cover positioned over at least a portion of the imaging module. The disposable cover may be removable. The disposable cover may be a single-use item.

The support arm may comprise a cylindrical casing.

The support arm may have a rigid construction.

The support arm may have a diameter less than or equal to 20 mm.

The angular orientation of the imaging unit may be fixed relative to the longitudinal axis. The at least one imaging unit may have a predetermined angular orientation. In a variant, the imaging unit may be rotatable about the longitudinal axis. For example, the imagining unit may be rotatable about the longitudinal axis.

The throat examination device may comprise a handle. The imaging unit may be disposed at a distal end of the support arm and the handle may be disposed at a proximal end of the support arm.

The handle may comprise a central plane. The central plane of the handle may be coincident with the longitudinal axis of the support arm. The first optical axis of the first lens and/or the second lens may be disposed in the central plane. This arrangement may facilitate manipulation of the throat examination device.

The support arm may be fixedly mounted to the handle, or may be formed integrally with the handle. Alternatively, the support arm may be removably mounted to the handle. The support arm or a portion thereof may be disposable. The support arm may, for example, comprise a disposable outer cover or casing.

The support arm may be less than or equal to 200 mm. This is appropriate to enable the imaging unit to be positioned at or proximal to the back of the patient’s throat whilst the controlling hand remains in the user’s line of sight and roughly approximated with the contralateral hand that is depressing the tongue. This may facilitate use and placement of the throat examination device.

The throat examination device may comprise one or more light sources. The or each light source may comprise a light emitting diode, for example. One or more light source may be provided on a lateral surface of the imaging module, for example adjacent to a lens of the imaging unit. Alternatively, or in addition, one or more light source may be disposed at a distal end of the throat examination device, for example a distal end wall of the imaging module.

According to a further aspect of the present invention there is provided a throat examination device for examining a throat of a patient, the throat examination device comprising:
a support arm having a longitudinal axis; and
an imaging unit comprising at least one imaging sensor for capturing an image of at least a portion of the laryngopharynx, the imaging unit being disposed on the support arm;
wherein the imaging unit has a transverse angle of view in a transverse plane extending perpendicular to the longitudinal axis of the support arm.

According to a further aspect of the present invention there is provided a throat examination device for examining a throat of a patient, the throat examination device comprising:
a support arm having a longitudinal axis; and
an imaging module comprising at least one imaging sensor for capturing an image of at least a portion of one or more of: the oral cavity, the oropharynx and the laryngopharynx;
wherein the imaging module is removably mounted on the support arm.

The throat examination device may comprise one or more mechanical fastener for releasably fastening the imaging module to the support arm. The or each mechanical fastener may, for example, comprise a cooperating male and female fasteners. The male and female fasteners may comprise cooperating threaded fasteners.

According to an aspect of the present invention there is provided a throat examination device for examining a throat of a patient, the throat examination device comprising:
a support arm having a longitudinal axis; and
an imaging module comprising at least one imaging sensor for capturing an image of at least a portion of the laryngopharynx;
wherein the imaging module is removably mounted on the support arm.

According to an aspect of the present invention there is provided a throat examination device for examining a throat of a patient, the throat examination device comprising:
a support arm having a longitudinal axis; and
an imaging unit comprising at least one imaging sensor for capturing an image, the imaging unit being disposed on the support arm. The imaging unit may be configured to capture an image of at least a portion of the oral cavity and/or the pharynx. The imaging unit may be configured to capture an image of at least a portion of one or more of the following:
   the oral cavity, the oropharynx and the laryngopharynx.

The imaging unit may comprise or consist of a spherical lens. Alternatively, the imaging unit may comprise at least one part-spherical lens. The or each part-spherical lens may, for example, be a hemispherical lens. For example, the imaging unit may comprise first and second part-spherical lenses. The first and second part-spherical lenses may be oriented in first and second directions, the first and second directions being opposite to each other.

The imaging unit may be configured to capture a wide-angle (panoramic) image, for example having an angle of view greater than 180°. The imaging unit may be fixedly mounted to the support arm, or may be provided in an imaging module which is removably mounted to the support arm.

The imaging unit may comprise or consist of one lens. An imaging sensor may be associated with the lens.

Alternatively, the imaging unit may comprise or consist of a first lens and a second lens. The imaging unit may comprise a single imaging sensor associated with both the first and second lenses. A light guide or optical device may be provided to guide light from the first and second lens to the associated imaging sensor(s). Alternatively, the imaging unit may comprise first and second imaging sensors associated with the first and second lenses respectively.

At least one of the first and second lenses may comprise a wide-angle lens or an ultra-wide-angle lens. At least one of the first and second lenses may comprise a fisheye lens. The first and second lenses may each comprise a wide-angle lens or an ultra-wide-angle lens. The first and second lenses may each comprise a so-called fisheye lens.

The imaging unit may have a transverse angle of view in a transverse plane extending perpendicular to the longitudinal axis of the support arm. The first lens may have a first subsidiary transverse angle of view greater than or equal to 180°; and/or the second may have a second subsidiary transverse angle of view greater than or equal to 180°.

The first and second lenses may comprise an overlapping field of view. The images captured by the first and second image sensors may comprise an overlapping image region. An electronic processor may be provided to process the captured images to form a composite image. The processing of the images may comprise reducing or removing the overlapping image region. The composite image may be formed which at least partially removes the overlapping image region. The image generated by the imaging unit may be a composite image formed by combining subsidiary images captured by two or more image sensors.

The imaging unit may be disposed at a distal end of the support arm. A handle may be disposed at a proximal end of the support arm. The imaging unit and/or the handle may be removably mounted to the support arm.

According to a further aspect of the present invention there is provided a disposable cover for covering at least a portion of an imaging module of a throat examination device. The disposable cover may be a single-use item. The disposable cover may be formed from a transparent material, such as a plastics material, to enable operation of one or more imaging units when the disposable cover is in place. Alternatively, or in addition, the disposable cover may comprise one or more windows for positioning over an associated imaging unit. The disposable cover may comprise one or more straps or tapes for fastening the disposable cover in place. The straps or tapes may each comprise fastening means, for example in the form of a mechanical fastener.

The imaging unit has been described herein as comprising one or more lens. Alternatively, or in addition, the imaging unit may comprise a prism or a light guide for directing incident light onto one or more imaging sensor.

Any control unit or controller described herein may suitably comprise a computational device having one or more electronic processors. The system may comprise a single control unit or electronic controller or alternatively different functions of the controller may be embodied in, or hosted in, different control units or controllers. As used herein the term “controller” or “control unit” will be understood to include both a single control unit or controller and a plurality of control units or controllers collectively operating to provide any stated control functionality. To configure a controller or control unit, a suitable set of instructions may be provided which, when executed, cause said control unit or computational device to implement the control techniques specified herein. The set of instructions may suitably be embedded in said one or more electronic processors. Alternatively, the set of instructions may be provided as software saved on one or more memory associated with said controller to be executed on said computational device. The control unit or controller may be implemented in software run on one or more processors. One or more other control unit or controller may be implemented in software run on one or more processors, optionally the same one or more processors as the first controller. Other suitable arrangements may also be used.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the present invention will now be described, by way of example only, with reference to the accompanying figures, in which:

FIG. 1A shows a schematic representation of a throat examination device in accordance with an embodiment of the present invention;

FIG. 1B shows a schematic representation of the throat examination device shown in FIG. 1A without the imaging module in place;

FIG. 2 shows a schematic representation of a control unit for the throat examination device shown in FIG. 1;

FIG. 3A shows an axial end elevation of the support arm of the throat examination device shown in FIG. 1;

FIG. 3B shows a side elevation of a distal end of the support arm of the throat examination device;

FIG. 3C shows a side elevation of a proximal end of the support arm of the throat examination device;

FIG. 4A shows a plan elevation of a handle of the throat examination device shown in FIG. 1;

FIG. 4B shows a side elevation of the handle of the throat examination device shown in FIG. 1;

FIG. 5A shows a first end elevation of the imaging module of the throat examination device shown in FIG. 1;

FIG. 5B shows a second end elevation of the imaging module of the throat examination device shown in FIG. 5A;

FIG. 5C shows a plan elevation of the imaging module shown in FIG. 5A;

FIG. 5D shows a side elevation of the imaging module shown in FIG. 5A;

FIG. 6A shows a first end elevation of the disposable cover for removably fitting over the imaging module of the throat examination device shown in FIG. 1;

FIG. 6B shows a plan elevation of the disposable cover shown in FIG. 6A;

FIG. 6C shows a side elevation of the disposable cover shown in FIG. 6A;

FIG. 7A illustrates the positioning of the throat examination device according to an embodiment of the present invention in an oral cavity of a patient;

FIG. 7B illustrates the field of view of the throat examination device shown in FIG. 7A;

FIG. 8 illustrates the positioning of a flexible endoscope through a nasal cavity of a patient;

FIG. 9 shows an end view of a variant of the imaging module shown in FIG. 5A in which the first and second lenses partially overlap with each other;

FIG. 10A shows a side elevation of the imaging module of the throat examination device according to a further embodiment of the present invention;

FIG. 10B shows an end elevation of the imaging module of the throat examination device shown in FIG. 10A; and

FIG. 10C shows a side elevation of the imaging module shown in FIG. 10A illustrating the field of view.

### DETAILED DESCRIPTION

A throat examination device 1 in accordance with an embodiment of the present invention will now be described with reference to the accompanying figures. The throat examination device 1 is an oral cavity insertion instrument adapted to be inserted into an oral cavity of a patient. The throat examination device 1 is suitable for examining the laryngopharynx (laryngeal pharynx), for example. At least in certain embodiments, the throat examination device 1 may be suitable for examining the oral cavity and/or the pharynx.

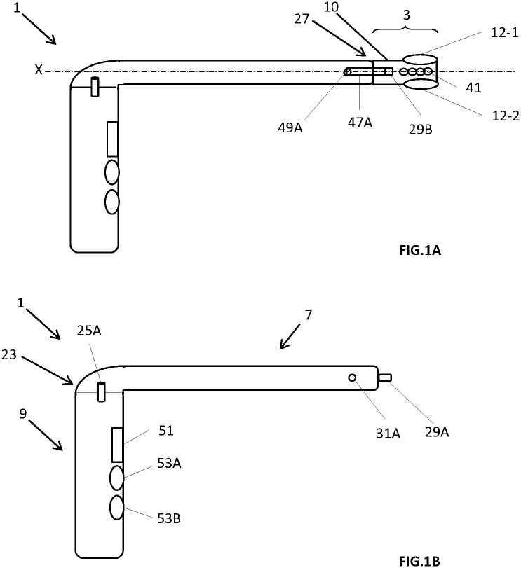

As shown in FIGS. 1A and 1B, the throat examination device 1 comprises an imaging module 3, a control unit 5, a support arm 7 and a handle 9. The imaging module 3 comprises an imaging unit 10 for capturing an image IMG of at least a portion of the laryngopharynx. The imaging module 3 forms a camera head for the throat examination device 1. The imaging unit 10 comprises at least one imaging sensor 11-n and one or more lens 12-n. The or each imaging sensor 11-n comprise an electronic sensor and may, for example, comprise a charge-coupled device (CCD) or an active-pixel sensor (CMOS sensor). The at least one imaging sensor 11-n is configured to generate image data IMD-n which is output to the control unit 5. The image data IMD-n may comprise a photographic (static) image and/or a video (dynamic) image. In the present embodiment, the imaging module 3 comprises a first imaging sensor 11-1 associated with a first lens 12-1; and a second imaging sensor 11-2 associated with a second lens 12-2. The first and second imaging sensors 11-1, 11-2 are configured to capture two images which are composited to form the image IMG for output. The first and second imaging sensors 11-1, 11-2 may, for example, output first and second image data IMD-1, IMD-2 respectively. The imaging module 3 is detachable from the support arm 7. In a variant, the imaging module 3 may be fixedly mounted to the support arm 7, or may be formed integrally with the support arm 7.

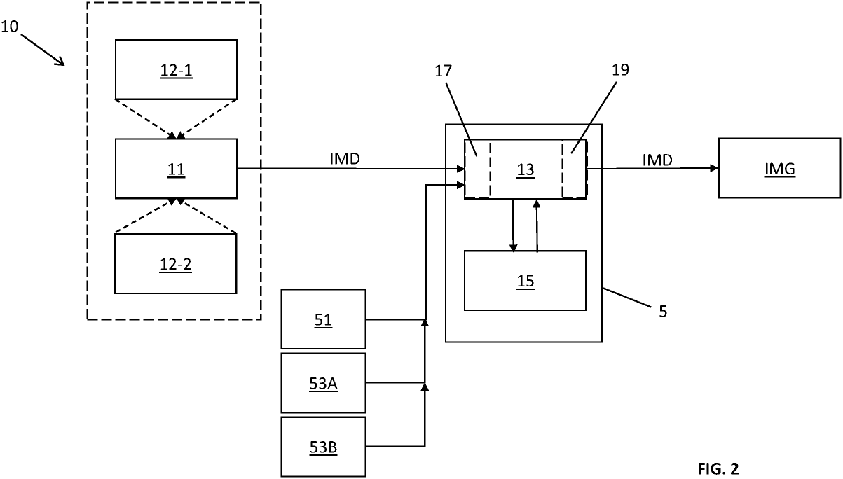

As shown in FIG. 2, the control unit 5 comprises at least one electronic processor 13 and a memory device 15. The at least one electronic processor 13 has at least one electrical input 17 for receiving the image data IMD-1, IMD-2 generated by the first and second imaging sensors 11-1, 11-2. The at least one electronic processor 13 is configured to generate composite image data IMD-C by compositing the first and second image data IMD-1, IMD-2 captured by the first and second imaging sensors 11-1, 11-2. The at least one electronic processor 13 has at least one electrical output 19 for outputting the composite image data IMD-C, for example to an external processing unit or a storage device. The composite image data IMD-C may be output for display on a terminal or screen 20 connected to the throat examination device 1. The at least one electronic processor 13 is configured to execute a set of computational instructions stored on the system memory 15. When executed, the computational instructions cause the at least one electronic processor 13 to perform the method(s) described herein. The control unit 5 is disposed in the handle 9 in the present embodiment. In a variant, the control unit 5 could be disposed externally, for example in a separate processing unit.

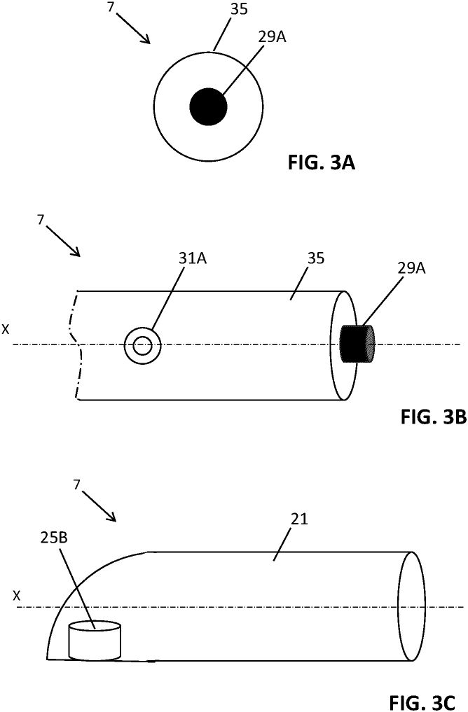

As shown in FIGS. 3A, 3B and 3C, the support arm 7 is generally cylindrical and has a central longitudinal axis X. The external diameter of the support arm 7 is approximately 15 mm. It will be understood that the external diameter of the support arm 7 may be less than or greater than 15 mm. An external diameter of less than approximately 20 mm is preferred. The total length of the support arm 7 (incorporating the handle 9) is approximately 200 mm. The diameter of the support arm 7 is substantially constant along the longitudinal axis X. The support arm 7 has a rigid (inflexible) composition such that the throat examination device 1 undergoes little or deformation or deflection under normal operating conditions. The support arm 7 comprises a casing 21 moulded from a plastics material. In the present embodiment, the casing 13 is a single-use item for disposal after one use. The casing 21 is replaced to ensure that the throat examination device 1 is sterile.

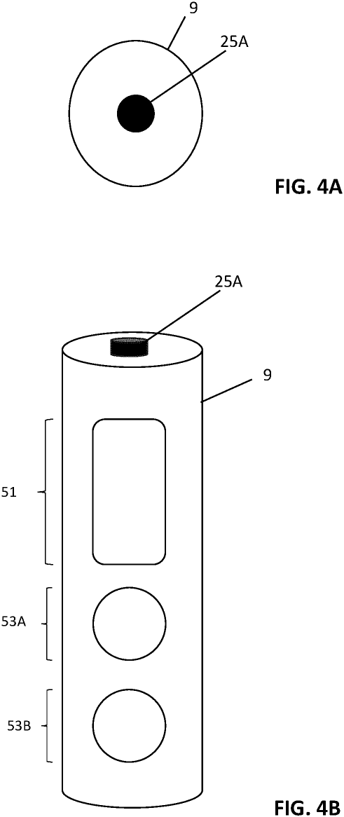

The throat examination device 1 comprises a first mounting assembly 23 for releasably mounting the support arm 7 to the handle 9. As shown in FIGS. 4A, 4B, the first mounting assembly 23 comprises cooperating first male and female connectors 25A, 25B. The first male connector 25A is mounted on a superior (upper) end of the handle 9 and comprises a shaft having an external thread. The first male connector 25A may, for example, be an insert mounted in the handle 9. The first male connector 25A in the present embodiment comprises a threaded shaft having a diameter of 5 mm. The first female connector 25B comprises an aperture having an internal thread for receiving the first male connector 23A. The first female connector 25B may comprise an insert, such as a threaded metal insert; or may be integrally moulded in a sidewall of the support arm 7. In the arrangement illustrated in FIGS. 2B and 3C, the first female connector 25B comprises a cylindrical insert having a diameter of approximately 10 mm. The first male and female connectors 25A, 25B extend substantially perpendicular to the longitudinal axis X of the support arm 7. In a variant, the first male connector 25A may be provided on the support arm 7. Other types of connector may be used. Alternatively, or in addition, the first mounting assembly 23 may comprise a locking mechanism (not shown) for locking the support arm 7 to the handle 5. By way of example, the locking mechanism may comprise a spring-biased member (such as a ball bearing) for engaging the support arm 7 to inhibit or restrict movement relative to the handle 9.

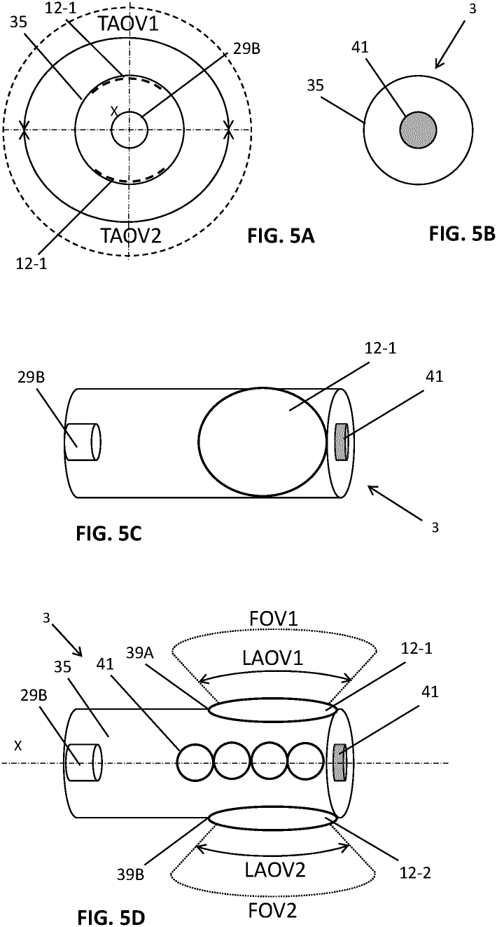

The imaging module 3 is removably mounted to the support arm 7. The throat examination device 1 comprises a second mounting assembly 27 for releasably mounting the imaging module 3 to the support arm 7. As shown in FIGS. 3B and 5C, the second mounting assembly 27 comprises cooperating second male and female connectors 29A, 29B. The second male connector 29A is disposed centrally on a distal end of the support arm 7. The second male connector 29A is in the form of a shaft having an external thread. The second male connector 29A may, for example, be an insert mounted in the support arm 7. The second male connector 29A in the present embodiment comprises a threaded shaft having a diameter of 5 mm. The second female connector 29B is disposed in a proximal end of the imaging module 3. The second female connector 29B comprises an aperture having an internal thread for receiving the second male connector 29A. The second female connector 29B may comprise an insert, such as a threaded metal insert, or may be integrally moulded in a sidewall of the support arm 7. The second male and female connectors 29A, 29B extend along the longitudinal axis X of the support arm 7. In a variant, the second male connector 29B may be provided on the support arm 7. Other types of connector may be used. First and second anchor points 31A, 31B are disposed on opposing sides of the distal end of the support arm 7. The first and second anchor points 31A, 31B are displaced from the distal end of the support arm 7 by approximately 30 mm in the present embodiment. Alternatively, or in addition, the first mounting assembly 23 may comprise a locking mechanism (not shown) for locking the imaging module 3 to the support arm 7. The locking mechanism may, for example, comprise a spring-biased member for engaging the imaging unit 4 to inhibit or restrict movement relative to the support arm 7.

The imaging module 3 comprises a central axis which is at least substantially aligned with the longitudinal axis X of the support arm 7. In a variant, the imaging module 3 may be inclined at an angle relative to the longitudinal axis X. The imaging module 3 comprises a housing 35 profiled to form a continuation of the support arm 7. The housing 35 has the same transverse section as that of the support arm 7. The housing 35 is generally cylindrical and has a diameter of approximately 15 mm and a length of 40 mm. The imaging module 3 comprises first and second lenses 12-1, 12-2 associated with the first and second imaging sensors 11-1, 11-2.

The first and second lenses 12-1, 12-2 are mounted in first and second lens apertures 39A, 39B formed in the housing 35. The first and second lenses 12-1, 12-2 in the present embodiment are oriented in opposite directions. In a variant, the first and second lenses 12-1, 12-2 mat be angularly and/or spatially offset from each other. The first lens 12-1 is disposed on a superior (upper) surface of the imaging module 3; and the second lens 12-2 is disposed on an inferior (lower) surface of the imaging module 3. The first lens 12-1 has a first optical axis Y1; and the second lens 12-2 has a second optical axis Y2. The first and second optical axes Y1, Y2 are aligned with each other and extend substantially perpendicular to the longitudinal axis X. As shown in FIG. 5D, the first and second lenses 12-1, 12-2 project radially outwardly beyond an outer wall of the housing 35.

The first lens 12-1 is a wide-angle lens having a first field of view FOV1. As shown in FIG. 5A, the first field of view FOV1 has a first (subsidiary) transverse angle of view TAOV1 in a plane extending perpendicular to the longitudinal axis X. As shown in FIG. 5D, the first field of view FOV1 has a first longitudinal angle of view LAOV1 in a plane extending along the longitudinal axis X. The first transverse angle of view TAOV1 defines an angular extent of the first field of view FOV1 in a circumferential direction around an exterior of the imaging module 3. The first longitudinal angle of view LAOV1 defines an angular extent of the first field of view FOV1 in a longitudinal direction.

The second lens 12-2 is a wide-angle lens having a second field of view FOV2. As shown in FIG. 5A, the second field of view FOV2 has a second (subsidiary) transverse angle of view TAOV2 in a plane extending perpendicular to the longitudinal axis X. As shown in FIG. 5D, the second field of view FOV2 has a second longitudinal angle of view LAOV2 in a plane extending along the longitudinal axis X. The second transverse angle of view TAOV2 defines an angular extent of the second field of view FOV2 in a circumferential direction around an exterior of the imaging module 3. The second longitudinal angle of view LAOV2 defines an angular extent of the second field of view FOV2 in a longitudinal direction.

In the present embodiment, the first transverse angle of view TAOV1 is approximately 180°; and the second transverse angle of view TAOV2 is approximately 180°. In a variant, the first and second transverse angles of view TAOV1, TAOV2 may each be greater than or equal to 160°. The first and second lens 12-1 are arranged such that the first and second fields of view FOV1, FOV2 do not overlap each other. The first and second transverse angles of view TAOV1, TAOV2 are subsidiary angles in this arrangement which cumulatively define the transverse angle of view of the imaging unit 10 in the transverse plane. The first and second lenses 12-1, 12-2 are arranged to provide a combined transverse angle of view AOV1 of approximately 360° about the longitudinal axis X. The first longitudinal angle of view LAOV1 may be in the range 90° to 135°; and the second longitudinal angle of view LAOV2 may be in the range 90° to 135°. In a variant, the first and second transverse angles of view TAOV1, TAOV2 may be greater than 180°. The first and second lens 12-1 in this arrangement have first and second fields of view FOV1, FOV2 which partially overlap each other.

The image IMG in the present embodiment is a composite image composed of at least a portion of each of the images within the first and second fields of view FOV1, FOV2 of the first and second lenses 12-1, 12-2. At least in certain embodiments, the image IMG has an angular extent of approximately 360° about the longitudinal axis X. Thus, the image IMG provides a substantially continuous (i.e. uninterrupted) representation of the region extending around the longitudinal axis X. In normal use, the image IMG may include a portion of the oral cavity and/or a portion of the patient’s throat. At least a portion of each of the images within the first and second fields of view FOV1, FOV2 may be stitched together to form the image IMG. The images may be cropped or otherwise transformed to form the image IMG.

A wireless connection between the imaging module 3 and the control unit 5 may be established using a suitable wireless connection. For example, a wireless connection may be established using Bluetooth (RTM), Wi-FI (RTM), or a cellular communication network (such as 4G or 5G). Alternatively, or in addition, a wired connection may be established between the imaging module 3 and the control unit 5.

The imaging module 3 in the present embodiment comprises a wireless (RF) transmitter T1 for transmitting the first and second image data IMD-1, IMD-2 to the control unit 5. The control unit 5 comprises a wireless (RF) transmitter R1 for receiving the first and second image data IMD-1, IMD-2. A short-range communication protocol may be used to transmit the first and second image data IMD-1, IMD-2. A processor (not shown) may optionally be provided in the imaging module 3 to control transmission of the first and second image data IMD-1, IMD-2. The processor may optionally process the first and second image data IMD-1, IMD-2 prior to transmission to the control unit 5, for example to generate the composite image IMG. In a variant, a wired connection may be established between the imaging module 3 and the control unit 5 for transmission of the first and second image data IMD-1, IMD-2. The control unit 5 is configured to process the image data captured by the at least one imaging sensor 11-n. The electronic processor 13 may perform image processing on the at least one set of image data IMD-n. For example, the electronic processor 13 may apply a mapping algorithm or a transform to the at least one set of image data IMD-n to compensate for optical distortions inherent in the first and second lenses 12-1, 12-2. The electronic processor 13 may correct image distortions caused by the first and second lenses 12-1, 12-2. The electronic processor 13 may apply a mapping algorithm or a transform. The processed image data may be combined to generate composite image data IMD-C. The electronic processor 13 may perform a cropping action and/or a transformation function to the first and second image data IMD-1, IMD-2. The control unit 5 may output the processed image data for storage and/or display on an external terminal. The external terminal may be local, for example having a direct (wired or wireless) connection to the throat examination device 1. Alternatively, or in addition, the external terminal may be remote, for example connected to the throat examination device 1 via the internet or other network connection. The displayed image IMG may be reviewed by a physician or other medical practitioner.

The imaging module 3 comprises one or more light source 41 for illuminating the oral cavity and throat of a patient. The one or more light source 41 comprise a plurality of light emitting diodes (LED). The one or more light source 41 may be disposed on one or both lateral sides of the imaging module 3; and/or at a distal end of the imaging module 3. The one or more light source 41 may be mounted so as to lie at least substantially flush with an outer surface of the housing 35. The imaging module 3 comprises a battery 43 (shown schematically in FIG. 5C) for supplying electrical power to the at least one imaging sensor 11-n and the light emitting diodes 41. The electrical connector 43 also provides a wired connection for transmission of the image data IMD-n1 from the at least one imaging sensor 11-n to the control unit 5. A cable (not shown) may optionally be provided in the support arm 7 to provide a wired connection for serial or parallel communication between the imaging module 3 and the control unit 5.

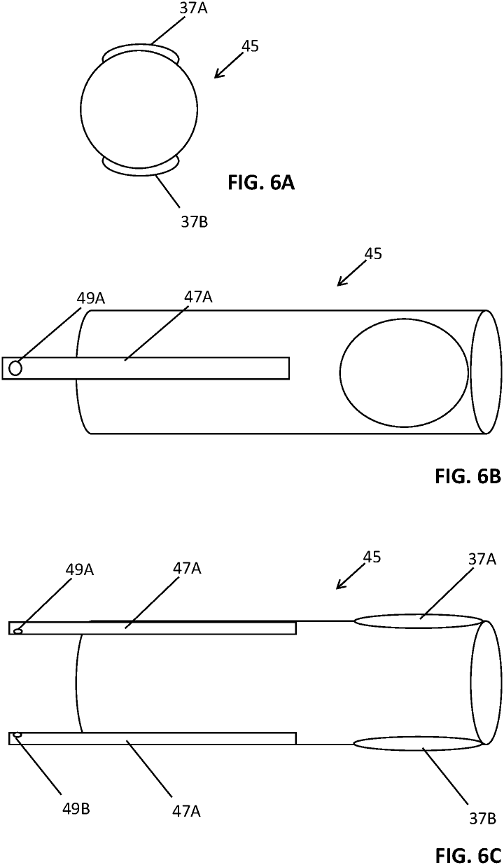

As shown in FIGS. 6A, 6B and 6C, a disposable cover 45 is provided to cover the imaging module 3 to prevent contamination. The disposable cover 45 is intended as a single-use item. The disposable cover 45 comprises a flexible material that fits over the imaging module 3. The disposable cover 45 is formed from a transparent material in the present embodiment. The disposable cover 45 is generally cylindrical and has a diameter of approximately 17 mm and a length of approximately 60 mm. The disposable cover 45 in the present embodiment comprises distended areas 37A, 37B that locate over the first and second lenses 12-1, 12-2. In a variant, the disposable cover 45 may comprise transparent windows for alignment with the first and second lenses 12-1, 12-2. The windows may be moulded from a transparent plastics material. The windows may be fastened to the disposable cover 45, or may be co-moulded with the remainder of the disposable cover 45. The disposable cover 45 comprises first and second straps 47A, 47B arranged to releasably fasten the disposable cover 45 to the support arm 7. The first and second straps 47A, 47B extend from a proximal end of the disposable cover 45 by approximately 10 mm. The first and second straps 47A, 47B cooperate with the first and second anchor points 31A, 31B provided on the support arm 7. The first and second straps 47A, 47B may, for example, comprise studs 49A, 49B locatable in respective first and second sockets forming the anchor points 31A, 31B. The studs 49A, 49B may be a snap-fit in the sockets, for example. Other fastening means may be used for retaining the disposable cover 45 in position. In a variant, the first and second straps 47A, 47B may comprise a releasable adhesive for adhering the disposable cover 45 to the support arm 7. The dual safety functions of the second mounting assembly 27 and the first and second straps 47A, 47B help to ensure the imaging module 3 is secured onto the support arm 7 and is not displaced during the examination. In a variant, the disposable cover 45 could be fastened to the support arm 7, for example to form a pocket into which the imaging unit 10 is inserted.

As outlined above, the support arm 7 is removably mounted to the handle 9. The handle 9 is disposed at the proximal end of the support arm 7 and is arranged to facilitate use of the throat examination device 1. In the present embodiment, the handle 9 is generally cylindrical in shape, having a diameter of approximately 4 cm and a length of approximately 125 mm to fit comfortably into a gripped hold. The handle 9 has a centreline which is oriented at least substantially parallel to an optical axis of the first lens 12-1. The centreline of the handle 9 is substantially perpendicular to the longitudinal axis X of the support arm 7 when the throat examination device 1 is assembled. Thus, the throat examination device 1 is generally L-shaped. The throat examination device 1 may comprise a curved bend between the support arm 7 and the handle 9 to improve ergonomic. This arrangement may facilitate placement of the imaging unit 10 for visualisation of the throat. At least in certain embodiments, an operator may position the imaging unit 10 using one hand.

As shown in FIG. 4B, the handle 9 comprises an image capture button 51 and two functions buttons 53A, 53B. The image capture button 5 in the present embodiment is generally rectangular and has a length of approximately 4 cm. The image capture button 51 is disposed on an anterior surface of the handle, approximately 1.5 cm below the superior (upper) surface of the handle 9. The image capture button 51 has a trigger-like action for controlling operation of the control unit 5. The image capture button 51 is used to control operation of the imaging module 3. For example, depressing and releasing the image capture button 51 may cause the control unit 5 to capture a photographic image; and depressing and holding the image capture button 51 for an extended period of time (for example greater than 0.5 seconds or 1 second) may cause the control unit 5 to capture a video image. The function buttons 53A, 53B are operable to change the settings of the control unit 5 and/or the imaging module 3. The function buttons 53A, 53B in the illustrated arrangement are circular and have a diameter of approximately 20 mm and are spaced apart from each other by approximately 10 mm.

The throat examination device 1 is assembled before the examination procedure starts. The sterile support arm 7 is screwed onto the reusable handle 9. The reusable imaging module 3 is then screwed onto the support arm 7. The disposable cover 45 is then placed over the display unit 3 and secured to the support arm 7. This arrangement ensures sterility of all parts of the device coming into contact with the patient (the display unit 3 and the support arm 7) whilst enabling the components with the highest associated cost (such as the handle 9 and the imaging module 3) to be reused. This also enables the invention to be reused rapidly by replacing the support arm 7 and the disposable cover 45 without the need to send the throat examination device 1 for sterilisation.

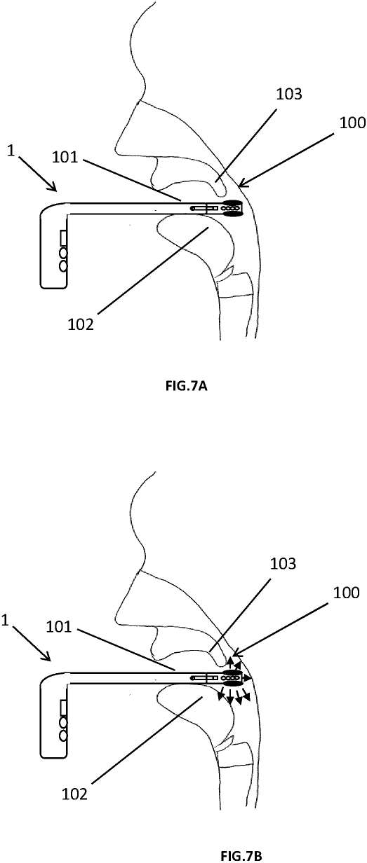

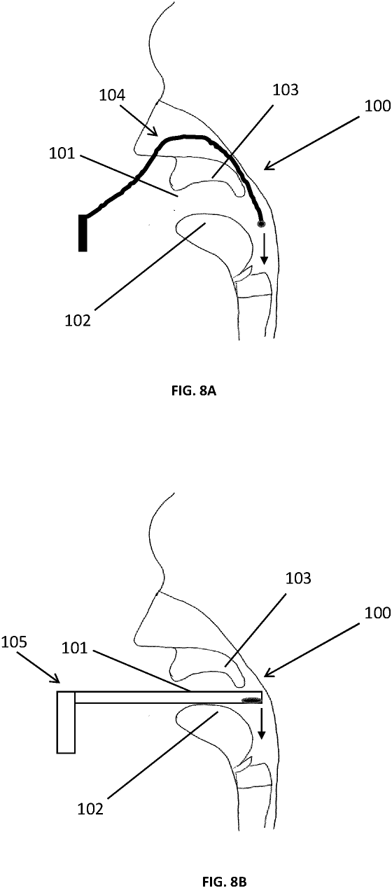

The use of the throat examination device 1 will now be described with reference to FIGS. 7A and 7B which shows a simplified diagram of the sagittal view of a throat 100, including the (oral cavity) mouth 101, tongue 102 and uvular 103. all highlighted. By way of comparison, a flexible endoscopic unit 104 is shown in FIG. 8A. The flexible endoscope passes through the nose and into the throat. The throat examination device 1 according to an embodiment of the present invention is illustrated in FIG. 7A. The throat examination device 1 is introduced into the mouth and the imaging module 3 positioned at a back part of the throat 10. The first and second fields of view FOV1, FOV2 are illustrated in FIG. 7B by arrows extending outwardly from the first and second lenses 12-1, 12-2.

The throat examination device 1 described herein generates a wide-angle image IMG of the mouth and throat of a patient. At least in certain embodiments, the image IMG may extend through 360°. The throat examination device 1 may be used single-handedly with the second hand being used to depress the tongue in order to access the back of the throat. The relative orientation of the support arm 7 and the handle 9 are designed to enable the imaging module 3 to be placed in the back of the throat under direct vision whilst maintain a secure grip on the throat examination device 1. The length of the support arm 7 is designed so the handle 9 will be in a equivalent position to the hand depressing the tongue at the point the imaging module 3 reaches the back of the throat and remains in the users line of sight. This helps with placement of the imaging module 3 and the ease of image capture. The size and positioning of the image capture button 51 on the handle 9 are designed to enable image capture whilst maintaining direct visualisation of the imaging module 3. The light sources 41 provide local illumination within the throat for image capture. The first and second lenses 12-1, 12-2 are disposed at the distal end of the throat examination device 1 and arranged opposite each other to enable image capture around the circumference of the imaging module 3. The width and shape of the imaging module 3 and the support arm 7 are designed so as to avoid the initiation of a gag reflex when the imaging module 3 is placed at the back of the throat.

The throat examination device 1 can be used to obtain images of the mouth, upper throat, and deep throat. These images can be used in the diagnosis and clinical follow-up of all types of mouth and throat disease. The capture of a video image may facilitate assessment of dynamic movements of the throat, for example involved in the functions of breathing, swallowing and voice. These largely encompass recording movements of the vocal cords, laryngeal structures, tongue, and pharyngeal walls. The throat examination device 1 is suitable for capturing an image of at least a portion of the hypopharynx. Furthermore, the throat examination device 1 may be suitable for capturing an image of the pharynx and/or the oral cavity.

The digital images that encompass all areas of the throat enable a comprehensive assessment in three dimensions, for example using virtual reality hardware to aid in the diagnosis and follow-up of throat conditions. Whilst modern endoscopy units can acquire three dimensional images, the limited field of view means that these would need to be serially reviewed to achieve the same comprehensive assessment.

The throat examination device 1 may be used by a medical practitioner. However, at least in certain embodiments, the throat examination device 1 could be used by a patient to perform self-examination of their own throat. This is especially true in the upper part of the throat where the tonsils sit as the patient only need to place the camera into the mouth and not all the way to the back of the throat. The image data IMD-n captured by the imaging module 3 may be transmitted over a network, such as the internet, for review by a trained specialist. This will aid telemedicine in the remote diagnosis and follow-up of throat disorders in the community such as tonsillitis. There is also potential to use artificial intelligence to perform automated analysis of the image.

The first and second lenses 12-1, 12-2 may comprise an overlapping field of view. The images captured by the imaging sensors may comprise an overlapping image region. An electronic processor may be provided to process the captured images to form a composite image. The processing of the images may comprise reducing or removing the overlapping image region. The composite image may be formed which at least partially removes the overlapping image region. The image generated by the imaging unit may be a composite image formed by combining subsidiary images acquired by two or more lenses.

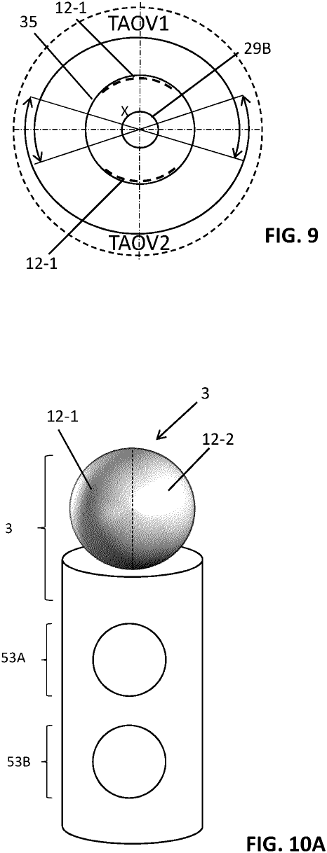

A further embodiment of the throat examination device 1 is shown in FIG. 9. The throat examination device 1 in this embodiment is a modification of the arrangement shown in FIG. 5A. Like reference numerals are used for like features.

The imaging module 3 in the present embodiment is a modified version of the unit shown in FIG. 5A. The imaging module 3 comprises first and second lenses 12-1, 12-2 associated with first and second image sensors respectively. The first and second image sensors are configured to capture first and second images which comprise an overlapping image region. The first lens 12-1 is a wide-angle lens having a first field of view FOV1. The first field of view FOV1 has a first (subsidiary) transverse angle of view TAOV1 in a plane extending perpendicular to the longitudinal axis X. The second lens 12-2 is a wide-angle lens having a second field of view FOV2. The second field of view FOV2 has a second (subsidiary) transverse angle of view TAOV2 in a plane extending perpendicular to the longitudinal axis X. The first and second transverse angles of view TAOV1, TAOV2 are subsidiary angles in this arrangement which cumulatively define the transverse angle of view of the imaging unit 10 in the transverse plane.

The first transverse angle of view TAOV1 is greater than 180°; and the second transverse angle of view TAOV2 is greater than 180°. In the present embodiment, the first transverse angle of view TAOV1 is approximately 180°; and the second transverse angle of view TAOV2 is approximately 180°. The first and second lens 12-1 are arranged such that the first and second fields of view FOV1, FOV2 partially overlap each other. The overlapping portion of each of the first and second images may be used to align the first and second images. For example, the image data IMD-1, IMD-2 may be processed to identify image elements or features which are common to both the first and second images. The image IMG may be formed by processing the image data IMD-1, IMD-2 associated with the first and second lenses 12-1, 12-2 at least partially to remove the overlapping image region.

The arrangement illustrated in FIG. 9 shows an overlap of the first and second fields of view FOV1, FOV2 in a transverse plane. The first and second transverse angles of view TAOV1, TAOV2 partially overlap with each other. Alternatively, or in addition, there may be an overlap in the first and second longitudinal angles of view LAOV1, LAOV2. The image IMG ma ybe formed by processing the image data IMD-1, IMD-2 associated with the first and second lenses 12-1, 12-2 at least partially to remove the overlapping image region.

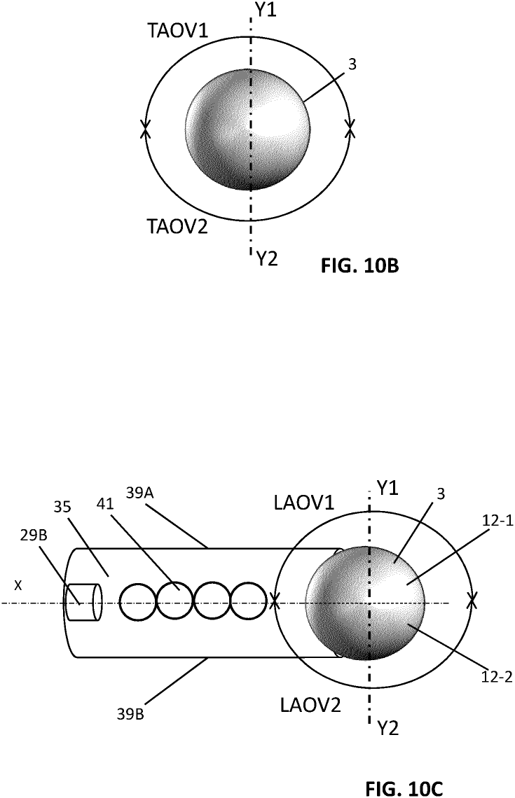

A further embodiment of the throat examination device 1 is shown in FIGS. 10A, 10B and 10C. The throat examination device 1 in this embodiment is a modification of the arrangement shown in FIGS. 5A to 5D. Like reference numerals are used for like features.

The imaging module 3 in the present embodiment is a modified version of the unit shown in FIG. 5A. The imaging module 3 in the present embodiment is at least substantially spherical. The imaging module 3 comprises first and second lenses 12-1, 12-2 associated with the first and second imaging sensors 11-1, 11-2. The first and second lenses 12-1, 12-2 each have a part-spherical field of view FOV1, FOV2. The first and second lenses 12-1, 12-2 each comprise or consist of a wide-angle lens in the form of a fish-eye lens. The first and second lenses 12-1, 12-2 are oriented in opposite directions. As shown in FIGS. 10A to 10C, the first and second lenses 12-1, 12-2 collectively have an at least substantially spherical profile. The first lens 12-1 is disposed on a superior (upper) side of the imaging module 3; and the second lens 12-2 is disposed on an inferior (lower) side of the imaging module 3. The first lens 12-1 has a first optical axis Y1; and the second lens 12-2 has a second optical axis Y2. The first and second optical axes Y1, Y2 are aligned with each other and extend substantially perpendicular to the longitudinal axis X.

The first lens 12-1 is a wide-angle lens having a first field of view FOV1. As shown in FIG. 10C, the first field of view FOV1 has a first (subsidiary) transverse angle of view TAOV1 in a plane extending perpendicular to the longitudinal axis X. The first field of view FOV1 has a first longitudinal angle of view LAOV1 in a plane extending along the longitudinal axis X. The first transverse angle of view TAOV1 defines an angular extent of the first field of view FOV1 in a circumferential direction around an exterior of the imaging module 3. The first longitudinal angle of view LAOV1 defines an angular extent of the first field of view FOV1 in a longitudinal direction.

The second lens 12-2 is a wide-angle lens having a second field of view FOV2. As shown in FIG. 5A, the second field of view FOV2 has a second (subsidiary) transverse angle of view TAOV2 in a plane extending perpendicular to the longitudinal axis X. The second field of view FOV2 has a second longitudinal angle of view LAOV2 in a plane extending along the longitudinal axis X. The second transverse angle of view TAOV2 defines an angular extent of the second field of view FOV2 in a circumferential direction around an exterior of the imaging module 3. The second longitudinal angle of view LAOV2 defines an angular extent of the second field of view FOV2 in a longitudinal direction.

The first and second transverse angles of view TAOV1, TAOV2 are subsidiary angles in this arrangement which cumulatively define the transverse angle of view of the imaging unit 10 in the transverse plane. The first and second lenses 12-1, 12-2 are arranged to provide a combined transverse angle of view AOV1 of approximately 360° about the longitudinal axis X.

In the present embodiment, the first transverse angle of view TAOV1 is greater than or equal to 180°; and the second transverse angle of view TAOV2 is greater than or equal to 180°. The first and second lens 12-1 are arranged such that the first and second fields of view FOV1, FOV2 do not overlap each other. Alternatively, the first and second lens 12-1 may be arranged such that the first and second fields of view FOV1, FOV2 partially overlap each other.

The first longitudinal angle of view LAOV1 may be in the range 135° to 180°; and the second longitudinal angle of view LAOV2 may be in the range 135° to 180°. In a variant, the first and second longitudinal angles of view LAOV1, LAOV2 may be greater than 180°. The first and second lens 12-1 in this arrangement have first and second fields of view FOV1, FOV2 which partially overlap each other.

The image IMG is a composite image composed of at least a portion of each of the images within the first and second fields of view FOV1, FOV2 of the first and second lenses 12-1, 12-2. At least in certain embodiments, the image IMG has an angular extent of approximately 360° about the longitudinal axis X. Thus, the image IMG provides a substantially continuous (i.e. uninterrupted) representation of the region extending around the longitudinal axis X. In normal use, the image IMG may include a portion of the oral cavity and/or a portion of the patient’s throat. At least a portion of each of the images within the first and second fields of view FOV1, FOV2 may be stitched together to form the image IMG. The images may be cropped or otherwise transformed to form the image IMG.

In certain arrangements the fields of view FOV1, FOV2 of the first and second lens 12-1, 12-2 may partially overlap each other (in a longitudinal direction and/or a transverse direction). A composite image may be formed by processing the images associated with the first and second lenses 12-1, 12-2 to remove the overlapping image region. A predefined algorithm may be defined to perform the image processing, for example to crop or otherwise transform a predefined region of each image. Alternatively, or in addition, the image processing may be performed dynamically. The overlapping portion of each of the first and second images may be used to align the first and second images, for example by identifying image elements which are common to both the first and second images.

In a variant, the first lens 12-1 may be disposed on a distal end of the imaging module 3; and the second lens 12-2 may be disposed on a proximal end of the imaging module 3. The first and second optical axes Y1, Y2 may be at least substantially aligned with the longitudinal axis X. In a further variant, the first and second optical axes Y1, Y2 may be angularly and/or spatially offset from each other. The second lens 12-2 could optionally be omitted. For example, the imaging module 3 may consist of one lens 12-1 disposed in a distal location. The lens 12-1 may be a wide-angle lens having an optical axis Y1 substantially aligned with or parallel to the longitudinal axis X.

It will be appreciated that various modifications may be made to the embodiment(s) described herein without departing from the scope of the appended claims.

The imaging unit 10 may be configured to determine a distance from the longitudinal axis X to an interior surface of the oral cavity and/or throat of the patient. The measured distance data may be used to generate a three-dimensional model. The image IMG captured by the imaging unit 10 may be mapped onto the model. Alternatively, or in addition, the model may be used to determine a position and/or an orientation of the imaging unit 10 within the oral cavity 3. The determined position and/or orientation of the imaging unit 10 may be used to generated feedback to ensure appropriate positioning of the throat examination device 1.

The imaging unit 10 has been described herein as having first and second lenses 12-1, 12-2 arranged to form a substantially continuous field of view FOV1. In a variant, the fields of view of the first and second lenses 12-1, 12-2 may be angularly separated from each other (in a transverse plane). For example, an angular separation of 45° or 90° may be present on each side. This arrangement may be appropriate if the lenses 12-1, 12-2 have a transverse angle of view which is less than 180°, for example.

Reference Numerals

| | |
|---|---|
| 1 | Throat examination device |
| 3 | Imaging module |
| 5 | Control unit |
| 7 | Support arm |
| 9 | Handle |
| 10 | Imaging unit |
| 11-n | Imaging sensor |
| 12-n | Lens |
| 13 | Electronic processor |
| 15 | Memory device |
| 17 | Electrical input |
| 19 | Electrical output |
| 21 | Casing (support arm) |
| 23 | First mounting assembly |
| 25A | First male connector |
| 25B | First female connector |
| 27 | Second mounting assembly |
| 29A | Second male connector |
| 29B | Second female connector |
| 31A | First anchor |
| 31B | Second anchor |
| 35 | Housing |
| 37A | First window |
| 37B | Second window |
| 39A | First lens aperture |
| 39B | Second lens aperture |
| 41 | Light source |
| 43 | Electrical connector |
| 45 | Disposable cover |
| 47A | First strap |
| 47B | Second strap |
| 49A | First stud |
| 49B | Second stud |
| 51 | Image capture button |
| 53A, 53B | Function button |
| 100 | Throat |
| 101 | Oral cavity (mouth) |
| 102 | Tongue |
| 103 | Uvular |
| 104 | Flexible endoscope |
| 105 | Rigid endoscope |
| IMG | Image |
| IMD-n | Image data |
| IMD-C | Composite image data |
| FOV1 | First field of view |
| LAOV1 | First longitudinal angle of view |
| TAOV1 | First (subsidiary) transverse angle of view |
| FOV2 | Second field of view |
| LAOV2 | Second longitudinal angle of view |
| TAOV2 | Second (subsidiary) transverse angle of view |

1-24. (canceled)

25. A throat examination device for examining a throat of a patient, the throat examination device comprising:
a support arm having a longitudinal axis (X), the support arm having a rigid construction;
a handle disposed at a proximal end of the support arm; and
an imaging module comprising an imaging unit disposed at a distal end of the support arm, the imaging unit comprising at least one imaging sensor for capturing an image of at least a portion of a pharynx and at least a portion of an oral cavity;
the imaging module having one wide-angle lens disposed in a distal location and having an optical axis substantially aligned with or parallel to the longitudinal axis;
wherein the angular orientation of the imaging unit is fixed relative to the longitudinal axis.

26. A throat examination device as claimed in claim 25, wherein the wide-angle lens is a spherical lens or a part-spherical lens.

27. A throat examination device as claimed in claim 25, wherein the wide-angle lens is a hemispherical lens.

28. A throat examination device as claimed in claim 25, wherein the imaging unit comprises a plurality of the imaging sensors.

29. A throat examination device as claimed in claim 25 comprising at least one control unit, the at least one control unit comprising:
an electronic processor having:
one or more electrical input for receiving image data generated by the at least one imaging sensor; and/or
one or more electrical output for outputting image data to an external device.

30. A throat examination device as claimed in claim 29 comprising a user interface for generating an image capture request in dependence on a user input, the at least one control unit being configured to capture image data generated by the at least one imaging sensor in dependence on the image capture request.

31. A throat examination device as claimed in claim 25, wherein the imaging module is removably mounted on the support arm.

32. A throat examination device as claimed in claim 31 comprising one or more mechanical fastener for releasably fastening the imaging module to the support arm.

33. A throat examination device as claimed in claim 25 comprising a cover for covering at least a portion of the imaging module.

34. A throat examination device as claimed in claim 25, wherein the support arm comprises a cylindrical casing.

35. A throat examination device as claimed in claim 25, wherein the support arm has a diameter less than or equal to 20 mm.

36. A throat examination device as claimed in claim 25, wherein the support arm is removably mounted to the handle.

37. A throat examination device as claimed in claim 25 comprising one or more light emitting devices disposed on the support arm and/or the imaging module.

38. A throat examination device for examining a throat of a patient, the throat examination device comprising:
a support arm having a longitudinal axis; and
an imaging module comprising at least one imaging sensor for capturing an image of at least a portion of one or more of: the oral cavity, the oropharynx and the laryngopharynx;
wherein the imaging module is removably mounted on the support arm.
